(19) United States
(12) Patent Application Publication
(10) Pub. No.: US 2006/0160883 A1
(43) Pub. Date: Jul. 20, 2006

(54) Regulation of immune system action in mammals through use of a multi-tiered model of immune system function

(76) Inventor: Linda M. Stoops, Harrisburg, SD (US)

Correspondence Address: Linda M Stoops;12th Street, 2407 West, Sioux Falls, SD, 57104, US

(21) Appl. No.: 11/192,936

(22) Filed: Jul. 29, 2005

Related U.S. Application Data
(60) Provisional application No. 60/643,866, filed on Jan. 14, 2005.

(51) Int. Cl. G06Q 10/00; A61K 31/405

(52) U.S. Cl. 514/419; 705/2

(57) ABSTRACT

This invention is a model of the mammalian immune system, having at least two tiers, and its use in the prevention, screening, testing, treatment and research of a variety of diseases and conditions, all of which produce the enzyme, indoleamine 2,3-dioxygenase (IDO) or inspire the body to produce IDO in reaction to them. Unlike the current model of the immune system that has a single tier in the decision process of whether or not the immune system will act,(Same (Self)/Different (Non-Self), this model takes the process at least a step further. Before any attack can occur, all targets judged Different must be judged at least once more. This decision is Baby or Not a Baby. No attack can occur unless the target is judged Different/Not a Baby. IDO production is the determining factor for this judgment.

### CROSS-REFERENCE TO RELATED APPLICATIONS

This application claims the benefit of Provisional Patent Application No. 60/643,866 filed Jan. 14, 2005 by the present inventor.

### FEDERALLY SPONSORED RESEARCH Not Applicable

### SEQUENCE LISTING OR PROGRAM Not Applicable

### BACKGROUND OF THE INVENTION

1. Field of Invention

This invention spells out a process for designing prevention, screening, testing, medical treatment, and research of diseases and conditions that involve production of the enzyme, indoleamine 2,3-dioxygenase (IDO) in mammals.

2. Prior Art

The 1998 announcement that scientists in Georgia had discovered the means by which fetuses escape attack by their mother's immune system (Munn, et al., Science, Aug. 21, 1998, 281(5380): 1191-1193) was fascinating for several reasons. Not only did the understanding of the enzyme, indoleamine 2,3-dioxygenase (IDO)'s role in fetal protection solve a historic mystery, but the whole situation highlighted a basic flaw in the immune system model science operates under today.

Biologically speaking, the goal of life is to survive long enough to reproduce and protect the young until they are able to fend for themselves and survive long enough to reproduce and so on. Yet, under the current model of immune function, protecting the fetus from the mother's immune system was, for years, a mystery. Now, with the understanding of IDO's role, this protection is seen as an exception to a rule or a fluke, if you will.

The current immune system model is based on a single tier. The question of whether or not the immune system will take action is based on a single determination: Is the target in question the same or different genetically from the immune system cell? Put another way-is the target Self or Non-Self?

Being judged Same (or Self), means there will be no attack. If on the other hand, the target is Different (Non-Self), the target will be attacked. Those judged Different would include, for example, mutated or damaged cells from the same body, bacteria, cells from different bodies (as in transplants), and even foreign objects like glass or wood splinters.

With this view in mind, it is easy to see the safety of the genetically-different fetus as a classic mystery. The same holds true for things such as cancer, AIDS, Parkinson's Disease, autism and a collection of other diseases and conditions. What is happening with regard to the immune system? This model has no answer.

Using the current model, scientists are at a loss to explain why the immune system does not take action in many diseases and conditions, so they believe the immune system is flawed. One researcher commented the “chronic infections, auto immune disorders, and the pathologic state of tolerance displayed toward tumors all suggest a breakdown in these fundamental immunoregulatory processes.” (Mellor, et al., The Journal of Immunology, 2003, 170: 5809-5813).

Besides explaining things, models also influence treatment choices. In the case of cancer, for instance, the current model sees the immune system's lack of attack as a flaw, and tries to either correct or work around it. Those who see the lack of involvement as permanent, are left to do the immune system's job themselves through surgery, gene therapy, chemotherapy or radiation. If one believes the immune system is asleep, interferon is an attempt to arouse it to attack, although indiscriminately. Some feel the immune system requires education and try vaccines to “teach” the immune system what to attack.

Other people have tried other ways to deal with the lack of immune system participation.

Linus Pauling proposed using high doses of Vitamin C to protect the body from outside attack or damage by building up collagen levels. (Naidu, Nutrition Journal 2003, Aug. 21, 2003, 2: 7; and Larsen, International Health News Database, International Journal of Alternative and Complementary Medicine, August 1997, 15 (8): 22-24). Collagen is a protein which is used to strengthen cell walls and makes up the tissue that holds the body together. The goal is to build a fortress, in effect, to protect the cells from damage and prevent cancer from spreading.

Dr. David Munn and Dr. Andrew Mellor, in Patent Application Number 20040234623 advocate using IDO inhibitors with other methods of treatment to treat cancer or infections (Munn, et al., U.S. Patent Application 20040234623, Nov. 25, 2004). This model would involve combining the IDO inhibitor with other treatments including chemotherapy, radiation, or cancer or viral vaccines.

According to a press release from the Medical College of Georgia dated Jul. 16, 2004, (www.sciencedaily.com/releases/2004/07/040716081345.htm), Dr. Munn and Dr. Mellor found that when they used IDO inhibitors in their study, the immune system “rallied”. It is unclear in their patent, then, why they would want to combine IDO inhibitors (to “rally” the immune system) with methods such as chemotherapy or radiation which would be damaging or destroying the immune system.

In general, there are a variety of problems with the current methods of cancer treatment.

Most are extremely expensive. Even with insurance, many patients are left with a lifetime of debt and, perhaps, bankruptcy for themselves or their survivors. According to the National Cancer Institute, 1 in 10 cancer survivors have $25,000 in cancer-related debt. (Marchione, AP/National Cancer Institute, published in the Sioux Falls Argus Leader, May 17, 2005: 3a).

Further, there is immediate physical damage for the patients. Hair loss, extreme illness, pain and nerve damage are among the side effects-effects so serious, in fact, that some prefer to not be treated at all or stop part way through rather than suffer for the chance the treatment will work. Quality of one's remaining time is an issue many consider.

For many others, there is no choice of treatment. Due to the type of cancer, the location of the tumors or the stage of the cancer, there is no treatment at all.

Finally, even those whose cancer is successfully treated are not safe from the continuing side effects. According to the National Cancer Institute, in a study of children who were diagnosed and treated for cancer and survived, the survivors were 2-6 times more likely than their healthy brothers and sisters to have health problems later in life. This damage was attributed to radiation and chemotherapy and included “heart disease, kidney problems requiring transplants or dialysis, blindness, infertility, mental retardation, paralysis, blood clots, lung problems, and even another cancer” (Marchione, AP/National Cancer Institute, published in the Sioux Falls Argus Leader, May 17, 2005: 3a).

In all, two out of three childhood cancer survivors later develop other chronic health problems caused by the cancer treatments they had (Marchione, AP/National Cancer Institute, published in the Sioux Falls Argus Leader, May 17, 2005: 3a).

Because current cancer treatments are not able to distinguish between “good” and “bad” cells, a lot of the “good” cells are killed with the “bad”, resulting in sickness, permanent, unintended damage and even death. Human efforts are inheritantly clumsy at doing the immune system's job.

Further, because the current model of immune system function leaves researchers Wrath only questions of why the immune system does not participate in the fight against cancer and a number of other diseases and conditions, the research has also suffered.

Despite billions of dollars spent on research since the National Cancer Act was passed in 1971, cancer is the biggest killer of Americans under 75 years old. An amazing 1.4 million Americans a year discover they have cancer (Leaf, Fortune, Mar. 22, 2004, 149 (6): 76+).

In terms of both treatment and research, the current immune system model has little to offer.

### OBJECTS AND ADVANTAGES

Instead of looking at the previously mentioned diseases and conditions through the view of the current, one-tiered model of immune function, a different point of vie v is in order. This is true not only in terms of it being an interesting theory, but also in the practical terms of how a different model translates to a method of choosing prevention strategies, screening and testing methods, treatments and directions to go with research.

The multi-tiered model proposed here properly explains immune function in those cases already understood, just as the one-tiered model does.

An advantage, however, is that it also explains situations that are currently mysteries, such as why the immune system does not attack cancer.

Further, this model ties into other known biological facts better than the one-tiered model.

For instance, both the existence of mammals and the protection of the unborn fetus from the mother's immune system are explained by the single-tiered model, in essence, as exceptions to a rule. This hardly seems to be the level of importance either example deserves. This is especially true when one considers that the second example, protection of a child, is part of the most fundamental of biological directives. As previously mentioned, the goal of biological life is to pass life to the next generation and so on. It is commonly held that the next generation is the overriding goal that much of life revolves around.

Since the mother's immune system is easily able to kill a fetus, thinking fetal protection would be a mere afterthought or fluke makes no sense.

With the proposed multi-tiered model, fetal protection is built into each and every decision regarding immune system attack. No attack could happen without the determination that a baby was not involved.

The one-tiered model concludes the immune system is flawed because it does not attack cancer (or given mutations, parasites, viruses, etc.)

Using the proposed multi-tiered model, one would conclude the immune system is alive, well, unflawed, and behaving exactly as it should based on the information it has. It is not flawed, just misled.

Furthermore, treatments based on this model would be drastically different than current practice.

Instead of trying to do the immune system's job through surgery, radiation, or chemotherapy, the goal would be to provide the immune system with the proper information to let it do its own job.

Instead of needing to “teach” the immune system with vaccines, the belief would be that the immune system already knows what to do; it just needs the proper information regarding its target.

Instead of promoting indiscriminate attack with interferon, this type of treatment would direct specific attack on only the “bad” cells.

Instead of believing stronger cells and connections achieved through more collagen is the final goal, treatments based on this model would try to give the immune system truthful information regarding its target.

Instead of using IDO inhibitors with treatments such as chemotherapy and radiation that damage or destroy the immune system, methods based on this model would view such combinations as either worthless and wasteful or actually harmful depending on the circumstances. Further, using this model, one would consider an IDO inhibitor combined with a vaccine to be both expensive and worthless since the goal in the multi-tiered model would not be to train the immune system, but to give the immune system the correct information so it can do what it already knows how to do.

While current treatments based on the one-tiered model are extremely expensive, many of those under the multi-tiered model are not. For instance, treating my dog for bone and cartilage cancer cost about $25 per month.

As mentioned earlier, many treatments in current use are not safe either in the short or long term.

Many of the treatments based on the multi-tiered model are known to be safe. Further, because the immune system is directed to specific attack under treatments based on this model, side effects resulting from immune system attacks on “good” cells are not a problem.

In the example of cancer, using treatments based on the current model is sometimes not possible. For instance, depending on the kind of cancer, its stage, and tumor location, many patients are left with no treatment options at all.

Because treatments based on this multi-tiered model are ones in which the immune system is doing the attack on a given target, this would, theoretically, not be as great an issue. The immune system is able to be present throughout the body and can work in places where other treatments cannot, so location of tumor or the fact it has spread would not be issues. Since all cancers are mutants, the type of the mutation would not be a factor. Mutated cells are a class of target the immune system regularly works on.

Further, because treatments based on this model normalize the levels of tryptophan and serotonin in the mammal being treated, depression, sleep problems, altered pain perception and other symptoms of low serotonin levels are alleviated. Descriptions such as feeling “fiesty”, or “spunky”, having energy to do things and feeling like one's self again are to be expected rather than the sickness, pain, and suffering associated with many current treatments.

Finally, while the one-tiered model has not led to the answers people have hoped for and worked for in so many of the diseases and conditions this patent refers to, having a new point of view for looking at them would be extremely helpful.

Having a new framework for research and understanding of the immune system's function in given circumstances may be the key to seeing the progress and results that are currently lacking.

### SUMMARY

This invention is a model of immune system function that uses more than one tier of questions to determine whether or not the immune system will act. Its use is to explain what is happening with the immune system in a given disease or condition and to make one able to choose the proper preventative measures, screening, testing, and treatments to start (or stop) an immune system attack.

### DRAWINGS

Not Applicable

### DETAILED DESCRIPTION--PREFERRED EMBODIMENT

Instead of a one-tiered decision regarding immune system function, this invention involves an immune system model with at least two-tiers for determining whether or not it will act.

In this model, the first tier is the same as in the other model, a determination of whether the target is the Same or Different. Same still would not result in attack. To be judged Different in this model, however, would not lead to attack. Instead, it would lead to the second tier where there is one more question: Is the target a Baby (Fetus), or Not a Baby (Not a Fetus)?

A judgment of Different/Baby, would not result in attack. The only way a target could be attacked in this model would be to be judged Different/Not a Baby.

At least one, and perhaps the only, determining factor for the immune system to decide whether a target is a Baby or Not is IDO. Indoleamine 2,3-dioxygenase, (IDO), as mentioned earlier, is the enzyme that protects a Baby from the mother's immune system. IDO acts by using O2 to catabolize tryptophan (a rare amino acid) (Fowkes, ForeFront-Health Investigations, July 1992). The mechanics of the fetus' protection may be: tryptophan decrease (Hwu, et al., The Journal of Immunology, 2000, 164: 3596-3599), increase in by-products of tryptophan catabolization such as kynurenine, 3-hydroxykynurenine and 3-hydroxanthranilic acid (Terness, et al., The Journal of Experimental Medicine, Aug. 19, 2002, 196 (4): 447-457; and Frumento, et al., The Journal of Experimental Medicine, Aug. 19, 2002, 196 (4): 459-468), indirect effects of IDO inhibition on vasoconstriction and macrophage activation (Kanellopoulos-Langevin, et al., Reproductive Biology and Endocrinology, 2003, 1: 121), or other, as yet unknown methods.

Regardless of the mechanics, IDO is the factor that determines whether or not the immune system attacks (Rutz, CNN, Aug. 21, 1998, www.cnn.com/HEALTH/9808/21/fetal.rejection/). It is at least one, if not the only, “Baby Sign”.

Using this multi-tiered model of immune system function, the goal of treatment would be to selectively expose the disease or condition in question to immune system action such as attack, repair, etc.

The process involved would be as follows:
1) Decide if this is a disease or condition in which IDO production is involved.
   This field is relatively new, but some cases are already known. For instance, as noted earlier, most cancer tumors studied so far produce IDO (Marshall, et al., BMC Biochemistry, 2001, 2: 5). Also, while I do not wish to be bound by this, I believe any disease or condition producing large amounts of oxidation will also be found to produce IDO.
2) For diseases or conditions that produce IDO, treatment would consist of a choice or choices from Part A below, and, as needed, a choice or choices from Part B and/or Part C below.
   Part A) A choice (or choices) that would include, but not be limited to, one or more types of antioxidants or a combination of antioxidants and one or more methods that stop or prevent production of indoleamine 2,3-dioxygenase (IDO), inhibit IDO, neutralize IDO or its action, or successfully compete with IDO and, so, stop it from acting. In short, this includes any method that turns off the IDO Baby Sign and allows immune system action.
   Choices include, but are not limited to: antioxidants (which compete with IDO for O2), or a combination of antioxidants and IDO inhibitors such as 1-methyl tryptophan, regulators such as iron levels, or other methods that accomplish the given goal.
   Part B) A choice (or choices) that would include nutrients, rest or any other method to build up the immune system. This would be especially important in cases in which the disease or condition is aggressive, the mammal's immune system is damaged from previous treatment or the disease/condition, or the mammal is in otherwise poor health.
   Part C) A choice (or choices) of an additional treatment that does not destroy, harm, diminish, or interfere with the immune system or its function, but does act on the targeted disease/condition or its effects. Cases in which this would be especially helpful include those in which the immune system is damaged or destroyed and one needs to buy time for it to recover, diseases/conditions which are too much for the immune system alone, etc.

Prevention strategies using this model would involve keeping the immune system as healthy as possible and keeping antioxidant levels at opitimal levels. Bowel tolerance would be a X ray of determining this level for an individual animal. (To determine bowel tolerance for ascorbic acid, one would give the mammal Vitamin C in increasing doses until the mammal gets diarrhea, then back down to the level at which the diarrhea stops.) Note, however, that these prevention methods would not be successful in cases of extreme injury, or very aggressive, fast-acting disease, for instance. In these cases, one would choose treatment strategies above.

Screening methods and testing for progress in treatment could be accomplished in several ways.

To screen for abnormal IDO production, or to check to see if the levels are going down, blood tests of tryptophan levels or kynurenine levels are used. Lowered levels of tryptophan or higher levels of kynurenine indicate higher levels of IDO.

While IDO is produced normally at several body locations, normal production levels are minimal (Terness, et al., The Journal of Experimental Medicine, Aug. 19, 2002, 196 (4): 447-457). These levels go up during pregnancy and with certain diseases and conditions (Gurevich, et al., Moscow State Medico-Stomatological University, Moscow, Russia, Dec. 27, 2000).

Because oxidation damage appears with the diseases/conditions under discussion, oxidation levels would also be useful information. Low levels of ascorbic acid in the blood, for instance, would indicate high levels of oxidation in the body. Also, the higher the bowel tolerance level, the higher doses of antioxidants one can tolerate. This would also indicated higher levels of oxidation are occurring.

For instance,blood serum levels of ascorbic acid are lower in people with viral infections than in healthy people (Naidu, Nutrition Journal 2003, Aug. 21, 2003, 2: 7). For another example, cancer patients have lower serum levels of ascorbic acid than people without cancer (Null, Townsend Letter for Doctors, February/March, 1994: and Cameron, et al., PubMed, 1:Proc Natl Acad Sci USA, October 1976, 73(10): 3685-9).

Although a number of diseases or conditions and treatment choices are possible, at this point, I will give an example of how this process works in one particular case.

The specifics in this case are as follows:

The mammal involved is Maggie, a 12 year old, neutered female Border Collie.
1) The IDO-producing diseases or conditions in this case are osteosarcoma (bone cancer) and chondrosarcoma (cartilage cancer).
2) There are three parts to treatment in this model. There must be a choice or choices from Treatment Part A. There may be choice(s) from Treatment Part B and/or Part C.
   A) The goal of Treatment Part A is to “turn off” IDO and/or its actions. The choice I used in this case was an antioxidant to compete with IDO for O2.
   The antioxidant I used was ascorbic acid in the form of Ester-C, a licensed trademark of Zila Neutraceuticals, Inc. It is manufactured under U.S. Pat. Nos. 4,822,816 and 5,070,085. It was purchased at Sam's Club and the bottle contained 500 500 mg caplets with Rose Hips.

The doseage was bowel tolerance level which in this case was 3-4½ grams per day.
B) The goal of Treatment Part B is to build up the immune system. Maggie was well-fed and in otherwise good health, so I did not focus on this part of the treatment.
I did, however, give her some health food store pills called Moducare for a short time.
Moducare is a registered trademark and is manufactured by Wakumaga of
America Company, LTD and by Essential Phytosterolins, Inc. It is supposed to balance and strengthen the immune system.
C) The goal of Treatment Part C is to choose any additional treatment(s) that acts on the disease or condition or its effects without destroying, harming, diminishing, or interfering with the immune system or its function.
One choice was amputation, since the tumor was breaking her leg bone and it could not be repaired. This is not, however, seen as a cure for either type of cancer she had.
Another choice was pain medication.
Yet another choice was antibiotics after the surgery to prevent infection while she healed.
My other treatment of choice was Vitamin D3. I purchased KAL brand D-400. (This stands for 400 IUs of Vitamin D3). I purchased them at several health food stores. There were 100 softgels per bottle and each softgel contained 400 IUs of D3 as well as 600 IUs of Vitamin A. They were manufactured by Nutraceutical Corp. for Makers of KAL, Inc.
Maggie's dosage most of the time was 6 softgels (2400 IUs) per day. For one week in November, she had no Vitamin D3, then after that, 10 (4000 IUs) per day for a short time, then 8 (3200 IUs) per day.
The total cost per month for Ester-C and D-400 was approximately $25.

Ideally, one would monitor blood levels for Vitamin D3 to check dosage amounts. Since Vitamin D3 is fat soluable, it can accumulate to dangerous levels in the body and cause other problems when the blood levels are too high, but it can also cause damage when the levels are too low. In this case, I may have given her too little.

Proper dosage of Vitamin D3 can also change depending on the amount of consistant sun exposure, type of cancer, how far it has spread, size of tumors, etc.

Note: Interferring with IDO production or activity in pregnant mammals can result in miscarriage or damage to the fetus.

Further, things that limit blood vessel growth (such as Vitamin D3) are not appropriate for use in pregnant mammals or for long term use in children or other young, growing mammals.

### OPERATION OF INVENTION

Under normal conditions, IDO is only produced in a few specific areas of the body.

As previously mentioned, the genetically Different mammal fetus produces IDO to ward off attack by the mother's immune system.

IDO is also produced in the eyes, guts and lungs (Marshall, et al., BMC Biochemistry 2001. 2: 5), all of which are places where the body is in constant contact with the outside world. In these instances, it is seen as an immune system regulator that prevents all-out attack every time a speck of dust hits the eye, for instance.

Besides normal production, certain “targets” produce IDO such as cancer tumors and certain parasites (Terness, et al., The Journal of Experimental Medicine, Aug. 19, 2002, 196 (4): 447-457; and Marshall, et al., BMC Biochemistry, 2001, 2: 5).

IDO is also produced in certain other situations.

Some feel it is used as a defense to kill invaders by starving them of the tryptophan they require (Marshall, et al., BMC Biochemistry, 2001.,2: 5; and Frumento, et al., The Journal of Experimental Medicine, Aug. 19, 2002, 196 (4): 459-468).

Others believe certain healthy cells are “recruited” by cancer cells to produce IDO (Medical College of Georgia Press Release, Jul. 16, 2004, www.sciencedaily.com/releases/2004/07/040716081345.htm).

Some even believe IDO is a cancer promoter (Hill, et al., ICI/FOCIS 2004, Abstracts, Number 2312, Publication Th53.122, Category Tumor Immunolgy).

Locating IDO producing cells and locations is a relatively new field of study.

While I do not wish to be bound by this, I believe they will find another cause for IDO production in many diseases and conditions. It is as follows:

There are two types of damage that happen in bodies.

One type is known as primary damage and includes cuts, poison, burns, impact injuries and other direct damage by injury or disease.

Some primary damage is limited (such as a cut), while other types of primary damage keep causing damage over a long period of time (such as Parkinson's Disease, MS, etc.)

Severity levels of primary damage range from the wear and tear of daily life to being blown to pieces by a bomb.

The more familiar elements of the immune system are designed to handle this sort of damage when possible.

Whenever primary damage occurs, secondary damage is also happening.

While the word, “secondary”, implies it is somehow not as important, except in cases of instant or nearly instant death, secondary damage is the actual mechanism of cell death.

Oxidation damage is the type of secondary damage this invention deals with.

O2, (superoxide anion) is formed daily as a waste product of cells. It also is formed when cells are damaged or killed.

Unlike regular oxygen (which is necessary for cell life), superoxide anion (O2) is a combination of 2 oxygen atoms which are missing electrons.

It is highly unstable.

Its combination with other molecules in the body causes problems. For instance, when it combines with H2, it creates H2O2 which is lethal to cells.

The body also has defense systems to deal with this sort of damage. The SOD enzymes and anti-oxidants from the diet or supplements are examples.

For a healthy person under normal circumstances, these defenses are sufficient, although, very slowly, the body is losing the fight since the aging process has ties to oxidation.

In some instances, however, the body's defenses are not sufficient to handle the amount of oxidation being produced. Examples would include bodies with a genetic inability to produce a SOD enzyme, low antioxidant levels in the body or an overwhelming amount of oxidation that the body can not keep up with. This may be from damage caused by severe infection or injury or from a slowly occuring accumulation of damage as in the cases of slow-moving disease or repeated smaller damage. Oxidation damages or kills cells and these actions produce more oxidation and so on.

At the point the body is confronted with more oxidation than it can deal with by other means, I think cells start producing IDO as a last resort to try to use up the oxidation before more cells are damaged or killed and more oxidation is created.

As previously mentioned, the enzyme, indoleamine 2,3-dioxygense (IDO) uses O2 to catabolize tryptophan.

In some cases, such as a sprain or surgical cut, the inflammation that sets off the IDO production slows the healing process, but protects more cells from being damaged or killed by the oxidation. In cases like this with primary damage of limited duration, this is a good defense strategy. Once the 02 is used up, there is no more need for IDO production, it stops and the immune system can get in to work on the primary damage.

In cases involving continuing primary damage, however, IDO production is not a good defense strategy.

In cases like cancer or MS, for instance, there is no limit to the primary damage and the resultant oxidation damage. Accordingly, IDO production and activity also continues.

Although IDO production and activity does save some cells from oxidation damage or death, it also keeps the immune system from being able to attack or repair the source of the primary damage.

In short, I think they will find IDO production in such cases is the body's last defense against oxidation damage and, while it buys the manual some time as it protects some cells from oxidation damage or death, in the case of continuing primary damage, it seals the mammal's fate by stopping the chance for the immune system to address the primary damage.

As mentioned before, scientists and doctors are at a loss to explain why the immune system does not attack in many diseases and conditions, so they believe the immune system is flaw ed.

Using a model with at least two tiers, one would pursue what it is about this target that is making it appear to be Different/Baby when it is not. Is it producing IDO? If so, the immune systems is doing what it is designed to do. It is protecting what it perceives to be a child. It is not flawed, just mislead.

Targets producing IDO would be judged Different/Baby and not attacked. Diseases and conditions that produce IDO then, would, in essence, be masquerading as babies and thereby escape immune system attack in this model's view.

A short list of diseases and conditions that may escape attack in this way includes cancer, HIV, group B strep, other persistent viral, bacterial, and parasitic infections, rheumatoid arthritis, Alzheimer's, depression, and others (Widner, et al., PubMed 1: J Neural Transm, 2000, 107 (3): 343-353; Marshall, et al., BMC Biochemistry, 2001, 2: 5; and Fowkes, ForeFront-Health Investigations, July, 1992).

If IDO is the “Baby Sign” as this model sees it, turning the sign off by stopping or blocking IDO and its action, should allow the immune system to attack. That is what happens. For example, in one experiment, inhibiting IDO led to immune system attack and miscarriage of mouse fetuses (Munn, et al., Science, Aug. 21, 1998, 281 (5380): 1191-1193). In another experiment, researchers said the immune system “rallied” against cancer cells when IDO was inhibited (Medical College of Georgia Press Release, Jul. 16, 2004, wwvw.sciencedaily.com/releases/2004/07/040716081345.htm.) It was suggested that “IDO is a cancer promoting” agent when cancer cells were attacked after IDO was inhibited in another experiment (Hill, et al., ICI/FOCIS 2004, Abstracts, Number 2312, Publication Th53.122, Category Tumor Immunology).

If IDO is the factor preventing immune system attack, the percentage of IDO being inhibited (or otherwise stopped, blocked, etc.) in a given case should be reflected in the degree to which the disease/conditions is affected. It is. For example, increasing the amount of inhibitor leads to increased T-cell proliferation. Additionally, immune response depended on the dosage of the inhibitor (Hwu, et al., The Journal of Immunology, 2000, 164: 3596-3599; and Hill, et al., ICI/FOCIS 2004 Abstracts, Number 2312, Publication Th53.122, Category Tumor Immunology).

Further, since the only way to be attacked in this model is to be judged Different/Not a Baby, turning off the “Baby Sign” by “turning off” IDO should result in specific immune attack instead of an indiscriminate attack. That is what happens. For instance, in the pregnant mouse experiment, upon inhibiting IDO, genetically different fetuses were attacked and killed by the mother's immune system, while fetuses genetically the same as the mother were not attacked (Kanellopoulos-Langevin, et al., Reproductive Biology and Endocrinology, 2003, 1: 121; and Marshall, et al., BMC Biochemistry, 2001, 2: 5).

As mentioned earlier, cancer is one of the diseases/conditions that produces IDO and, so, in this model's view, is being perceived as Different/Baby. Because of this judgment, there is no attack by the immune system. Treatment, then, would involve turning off the IDO “Baby Sign” by stopping its production, inhibiting, neutralizing it or its action, or successfully competing with it to stop it from acting or in any other way blocking IDO or its action. This would allow the immune system to get the proper information about the target and attack.

Not only would the immune system be able to act, but an added benefit of this treatment is that it leaves the individual with a sense of well-being. By stopping or eliminating IDO's action, it is not able to catabolize tryptophan. This leaves more tryptophan available for forming serotonin, a neurotransmitter that affects sleep, and perceptions of pain, relieves depression, etc.

Regarding Maggie's treatment, the main reason I chose Vitamin C (ascorbic acid) for Part A was that IDO uses superoxide anion to catabolyze tryptophan (Fowkes, ForeFront-Health Investigations, July, 1992; and Johnson, CPS: medichem/0105004, Independent Research, May 29, 2001) and Vitamin C is a superoxide anion scavenger (Naidu, Nutrition Journal, Aug. 21, 2003, 2: 7). Since they are competing for O2, adding Vitamin C would limit or eliminate the O2 the IDO needs to act, and, thereby, eliminate the need for further IDO production.

Further, Vitamin C: is non-toxic and safe (Null, Townsend Letter for Doctors, February/March 1994; Megascorbate Therapies: Vitamin C in Medicine: Vol 1,1, The Vitamin C Foundation, 1997; and Vojdani, et al., Immunosciences Lab, Inc. 2000), is readily available (Naidu, Nutrition Journal, Aug. 21, 2003, 2: 7), is inexpensive, is delivered systemically, can be administered in a variety of ways including orally, powder, IV, or by injection. Individualized dosage is easily arrived at and one cannot overdose on it, and there is a “buffered” form, Ester-C, that allows high doses without stomach upset. Vitamin C has other cancer-fighting properties besides blocking IDO (British Columbia Cancer Agency, Provincial Health Services Authority, February 2000), it can work in and on every cell in the body (Megascorbate Therapies: Vitamin C in Medicine: Vol 1,1, The Vitamin C Foundation, 1997; and Null, Townsend Letter for Doctors, February/March, 1994) and it does not weaken or interfere with immune system actions (Naidu, Nutrition Journal 2003, Aug. 21, 2003, 2: 7; and Null, Townsend Letter for Doctors, February/March, 1994).

Some reasons I chose Vitamin D3 for Treatment Part C were that it is non-toxic , readily available and inexpensive. Vitamin D3 works against cancer in several ways (Osborne, et al., Leicester Royal Infirmary, PubMed, 1: Br J Dermatol, August, 2002, 147(2): 197-213; and Diaz, et al., Cancer Research, Apr. 15, 2000, 60: 2304-2312) and it does not weaken or interfere with immune system actions.

Finally, (and the main reason I chose it), Vitamin D3 stops blood vessel growth (Angiogenesis Foundation: Pets & Wildlife-Canine, www.awgio.org/pets_and_wildlife/petcanine_cancer/osteo.html). Tumors require more glucose than cells surrounding them because of their rapid growth rate. To get what they need, they grow their own blood vessels. Primary tumors use this method to stop secondary tumor growth by releasing angiogenesis (blood vessel growth) inhibitors into the blood stream. For example, in canine osteosarcoma (bone cancer), until the primary tumor,( which is usually in a leg), is removed, the secondary cancer, (which is usually in the lungs or liver), does nothing.

More Information Regarding My Dog's Cancer

Maggie was 12 years, 6 months old when she started limping in mid-June, 2004. When the limp did not stop, the veterinarian prescribed antibiotics for a week. No improvement was seen. She was also started on prescribed pain medication.

A July 2nd x-ray showed bone cancer in her left, front leg. The bone was breaking from the pressure of the tumor pushing from the inside. The veterinarian said if one amputated the leg and did nothing else, the dog's life expectancy would be 3-5 months or around 12 months with chemotherapy. Apparently, by the time one notices limping, the cancer has already spread-usually to the lungs and liver. In general, it does not spread to other bones.

A July 2nd blood test report, showed an extremely high alkaline phosphatase level of 3175. The normal range on this test is 5-131.

A biopsy done days later showed 2 kinds of cancer: osteosarcoma and chondrosarcoma (bone and cartilage cancer). Both types are very aggressive.

On July 12, Maggie's leg was amputated.

Also, on July 12, she started taking Vitamin D3. (She started with 2 per day, and by July 14th, was taking 6 per day. She took 3 in the morning and 3 at night from then until October 30.) The hope was that the D3 could buy some time. I did not expect it would cure the cancer, but I hoped it would prevent the tumors from growing.

She also took Moducare (an immune system pill from the health food store.) She took 1-2 per day for 2 weeks.

Researchers at Colorado State University had found that high alkaline phosphatase levels like this subject's correlated with much shortened life expectancies. In a phone call, one of the researchers said that with such a high level, they would expect this dog to have half the life expectancey of others. (That would be one and one half months to two and one half months(mid August to mid-September) if not given chemotherapy after amputation and 6 months(December) if she was given chemotherapy after amputation.)

Further, the researcher said that their studies indicate that when the alkaline phosphatase level goes down, it indicates a treatment is working. When it goes up, it indicates the cancer is spreading.

By August 15, the dog was showing no signs of illness or weight loss, although she was still not as alert as normal. This had been increasing gradually for some time before the cancer was found and, originally, was thought to be caused by age. She slept alot and when I arrived at home at night, she would not awaken until I walked across a wood floor and called her name. Sheep dogs, and particularly Border Collies, do not normally behave this way.

X-rays taken August 15 showed no lung cancer tumors. Blood tests taken then showed the alkaline phosphatase level had dropped slightly from 3175 to 3113.

August 25, in addition to Vitamin D3, Maggie started taking Vitamin C (Ester-C caplets). That evening, she was given 3 caplets (one and one half grams).

The morning of August 26, she was given 4 caplets (2 grams). The goal was to find her bowel tolerance level.

That evening, Maggie was waiting at the door when I arrived at home. Instead of sleeping, she was alert and awake.

On August 29, I noticed Maggie was uncomfortably hot, panting, drinking extra water, and lying around as if she had a fever. It was theorized that her immune system would attack the cancer at this point, and so the fever and other conditions would be consistant with that theory.

By August 30, Maggie was getting back to her normal behavior. From that point, improvement was steady and dramatic. Each week, she improved in some way. At the beginning, she could walk only yards without resting. Each week she could go further and faster. After several weeks, she could run approximately 100 yards to the barn and back again without stopping to rest.

Maggie had always been a dominant dog, and during treatment, started taking other dog's bones and guarding them, and even barked at and chased the rare vehicle that passed her home. She even started jumping off a wooden floor on to a couch to nap.

On October 30, the dog's hind legs seemed shaky and each day of the following week, the hind legs seemed weaker. Fearing an overdose of Vitamin D3, I stopped giving it to her for a week.

A blood test taken November 3rd, surprisingly showed no signs of Vitamin D3 overdose according to the vet. It was thought the Vitamin D3 was being used as soon as it was administered. The same effect was seen with the Vitamin C. The test also showed her alkaline phosphatase level had dropped to 2230.

On November 5, she was weaker yet. Lung and lower abdominal x-rays showed no sign of cancer and no calcium deposits in any organs (a symptom of D3 overdose). She had not lost weight.

That evening, she was weaker yet and so I took her to the Vet Emergency Hospital. Blood tests there showed her alkaline phosphatase level to be 1669-a drop since two days previously. These tests were done by different labs, so the difference may not be significant.

On November 6, her lower back and hind legs were x-rayed. The x-ray showed some arthritis and a pinched nerve which explained the weakness and trouble walking. An examining vet believed the pinched nerve was caused by adjusting to walking on three legs at her age and that it would heal in two to three weeks.

The x-ray also showed a tumor in one hind leg bone. It had made a hole in the bone, but had not yet broken it. It was unclear whether this tumor was a dead or dying one or one that had started before or after her treatment had started, or a new one that had developed since the D3 had been stopped, but if still alive, it was expected to break the bone in the very near future,.

Treatment resumed, this time with 9 softgels of Vitamin D3 per day and 9 caplets of Ester-C per day. That afternoon, she developed a fever that lasted until afternoon of the next day.

A week later, she was taking 7 Ester-C and 10 D3 per day, but the D3 was dropped to 8 per day by November 21.

She gradually improved and by Thanksgiving week was able to lift herself from the ground and walk without assistance.

Later that week, she pinched another nerve and lost the use of her hind legs again and the control of her bladder and bowels.

By December 13, she could control her bowels and bladder and move both hind legs and her tail.

During the 2 week period before her death, her bowel tolerance level for ascorbic acid went down twice.

A week before her death, Maggie had spent the day in the garage. She had a hay bed and the temperature was in the 40's. The doors and windows were closed.

When I came home, I found the walk-in garage door wide open and a package on the front door step. A very cold wind was blowing in and I found Maggie lying flat on her side, off the hay, on the concrete floor. She was very still and did not move at all when I came in. I picked her up and carried her into the house, covered her with blankets, and put a hot water bottle on her. She was terribly cold, but had not moved herself onto the hay or away from the wind at the door. (She could not stand, but could scoot herself around with her front leg.)

During the next two nights, (December 10th and 11th), she was extremely restless and could not get comfortable. Her gums were very pale. She had been acting hotter than the other dogs, panting and drinking alot. She barked and whined all night both nights unless I sat right with her. She was urinating every hour. When I would finally get her into a comfortable position on the pillows and her dog bed, she would immediately fall into a deep sleep.

Sunday night, she was better and was only up at 3 a.m. and 6 a.m. By Monday, she was back to a more normal pattern of drinking and seemed much more comfortable by that evening. The color came back to her gums.

I wondered if she surprised the delivery person by barking and the person may have kicked or otherwise hurt her. After her death the next Saturday, we were trying to understand why there was cloned blood near her liver. Another thought was, perhaps I had hurt her by lifting her with a towel under her torso. She was too heavy for me to lift off the ground, so I usually had to move her by pulling her across the floor with a towel or lifting her with one.

Starting the evening of December 15, she did not move her hind legs or tail-possibly another pinched nerve.

The evening of December 17, she did not eat her food until fed by hand. This was a first.

The morning of December 18, she did not eat at all. That afternoon, she was alert and laying upright by the door waiting for me when I got home. After about twenty minutes with her, I left the room. After about 20 minutes, I came back into the room and found her dead.

Immediate cause of death appeared to be asphyxiation while vomiting. Because of the pinched nerve, she could not lift herself with her hind legs to get her head down.

An autopsy showed fluid in her lungs, but there was no cancer in her lungs.

The tumor discovered in her leg in early November never did break her bone. The tumor area was being replaced by a combination of living cells and dead cancer cells.

There was a good deal of cloned blood in her abdominal cavity near her liver, but the examiner could not find the place in her liver it had come from.

Cancer had spread to her liver at some point, but it was not said whether it was alive and active at the time of her death. It was limited to single cells and microscopic clusters.

She had fat damage to her liver.

The examiner did not find cancer in her spine, but there was an abnormally soft area. Such damage might have been caused by not enough Vitamin D3.

A blood clot at some point had damaged one half of one kidney.

An infection at some point had effected her heart valves.

She had some other age-related damage.

The most significant finding was that, while she w,as an older dog and had some age-related and other damage, she did not die of cancer. The only cancer cells found were the microscopic cells and clusters in her liver and the bone tumor area that was being replaced by the combination of dead cancer cells and living, healthy cells. The treatment seemed to be working on the cancer.

She would have been 13 years old in January.

### Description of Operation of Alternative Embodiments

There are many alternative embodiments of this invention.

They may be classified as follows:

A) Treatments for other diseases and conditions besides cancer

B) Different choices for Treatment Part A

C) Different choices for Treatment Part B

D) Different choices for Treatment Part C

E) Different methods for screening and testing for progress of a treatment

F) Different methods regarding turning on IDO production.

Regarding A): While cancer is the disease I have used for an example throughout this paper, as previously mentioned, there are a number of diseases and conditions associated with IDO production. Examples such as inflammation, depression, Alzeimer's Disease and possibly Parkinson's Disease, MS, Gulf War Syndrome, and autism, for instance, are not to be seen as a complete list, but they do give an idea of the scope of things being covered.

As previously mentioned, I also believe they will find that any disease or condition that overwhelms the body's defenses against oxidation damage will be found to involve IDO production as a last-ditch effort to stop that damage. While breaking down tryptophan may not be the immediate goal in such cases, IDO does use O2 to do this and in these cases, would stop O2 damage and death in the immediate area.

B) In the cancer example sited earlier, I chose antioxidants, specifically ascorbic acid, for the Treatment Part A choice. The caplet form of Ester-C was easy to give the dog and keep track of.

In the case of people who are being treated for cancer, Ester-C would still be a good choice, but it could be combined more easily with other antioxidants.

For instance, many foods contain antioxidants (such as oranges, broccoli, peppers, etc.) as do coffee, tea, and various other nutritional supplements. Other choices of antioxidants or combinations are acceptable choices as long as enough is taken to use up the available O2.

Because some antioxidants are fat soluable, dosage may be a bit harder to figure out than it would be for ascorbic acid. Also, while ascorbic acid can go anywhere in the body, it is not clear to me that all antioxidants can do that. Depending on the location of the target one is trying to treat, that would be a factor.

IDO inhibitors and iron levels are also possibilities for choices for Treatment Part A. Although each is able to “tie up” or regulate the IDO, they do not address the oxidation levels which may be instigating IDO production and which are definitely causing cellular damage or death.

Antioxidants would have the advantage of addressing both IDO production and action and oxidation damage. In practice, I think one would need to use some antioxidant with either IDO inhibitors or lowered iron levels. At first glance, it seems easier and more efficient to just go with the antioxidants, but further thought may present a reason for closing IDO inhibitors or lowered iron levels.

The same line of thought includes the use of genetic engineering to shut down IDO production. While it would work, theoretically, to shut down IDO production, I think one would still need to use antioxidants to stop the oxidation damage. Because of this, I am not sure it would be either practical or a better choice than using antioxidants alone.

C) Depending on the mammal being treated, taking specific action to build up the immune system may or may not be necessary. While any method(s) that builds up or strengthens the immune system is a suitable choice for Treatment Part B, but making no choice at all may also be appropriate.

D) Choices for Part C of this treatment would depend entirely on the case and the disease or condition being treated.

For instance, while Vitamin D3 would be one of the favored treatment choices for any of the diseases or conditions with relation to sun exposure (such as cancer and Parkinson's Disease), it may not be the best choice in other cases, such as severe burns.

Further, in the case of Vitamin D3, one may use all supplements, part sun exposure/part supplement, or, in some locations and times of year, all sun exposure. Another choice is items containing components the body uses for D3 production.

In any case, Vitamin D3 is not to be seen as a necessary choice for these treatments. Treatments for Part C may range from chosing nothing in some cases to a combination of many things or procedures. The only rule here is that choices that damage or destroy the mammal's immune system are not appropriate choices for optimum results.

For instance, a look at the combination of chemotherapy and antioxidants will better explain this thinking.

If the mammal starts taking sufficient antioxidants before the start of chemotherapy, the chemotherapy will be neutralized by the antioxidants. Chemotherapy does its work through oxidation damage and the antioxidants would cancel that action out. While there would be a slight damage from the primary damage of injesting a poison, the usual side affects one expects would not happen, such as damage or destruction of the immune system, hair loss, extreme illness, weakness, etc. In this case, taking the chemo with the antioxidants would be both expensive and generally worthless.

If the mammal starts taking antioxidants after the start of the chemotherapy, the antioxidants will still stop some of the oxidation damage, but one runs the risk that the immune system has already been heavily damaged or destroyed. This would be a dangerous and possibly lethal situation, since the model for this treatment is based on having the immune system act on the targeted disease or condition. In short, combining antioxidants with chemotherapy would not be a good choice.

The same thoughts would apply to radiation since radiation also does its work through oxidation damage (Naidu. Nutrition Journal 2003, Aug. 21, 2003, 2: 7).

E) Screening and testing for progress can be accomplished several ways.

Screening for high IDO levels by doing a blood test for low tryptophan levels or high kynurenine levels or by looking for low antioxidant levels would all be useful information. In terms of Vitamin C, serum ascorbic acid levels can be tested or the bowel tolerance method can be used in which one takes Vitamin C up to the level of diahrrea. High bowel tolerance would indicate an oxidation-producing disease or condition as would low blood levels of ascorbic acid which is often, if not always, an indicator of IDO production.

While these screening methods are not specific to which disease or condition is present, they would at least be a reason to look further.

During a successful treatment of this method, IDO levels should drop and ascorbic acid levels should rise in the blood. Under these new conditions, one would administer lower doses of IDO inhibitors (if using that choice) and lower doses of antioxidants. Testing methods would be as mentioned above for screening.

F) There are also different embodiments for cases in which one would want to “turn on” IDO production at a given site to prevent or stop immune system action. The goal in this case would be to have the target be perceived as either “Same” or “Different/Baby”, both of which are categories that do not come under attack. Turning on IDO in these cases would make the target be perceived as Different/Baby.

An example of a situation in which one would want to do this would be to protect a transplant from immune system attack and eliminate the need for anti-rejection drugs. Theoretically, one would not even need a transplant from another mammal of the same species. One could, perhaps, transplant pig hearts into humans, for instance.

Using a drug that focuses solely on the transplant or genetically engineering individual cells of an organ to produce IDO are the probable methods for this.

It seems this embodiment, while possible, would have a number of challenges.

For instance, IDO production would lead to lower tryptophan and serotonin levels. Issues such as depression, lack of sleep, altered pain perception and other areas affected by low serotonin levels would need to be addressed.

Further, the previously described method of treating cancer, for instance, would not be available to such patients since the treatment would also direct attack to the transplant.

On the positive side, however, if one can deal with or treat the serotonin-related issues, it would conceivably open the door for not only standard transplants, but even transplants between species without fear of rejection.

Advantages of this would include the lack of need for anti-rejection drugs with their related costs and health issues, but it also would drastically cut waiting lists for organs by opening up new classes of potential donors. For instance, if pig hearts could now be used in humans without being rejected, there would be no reason to wait for another human to die.

Even if these transplants could only be used on a short term basis, it would be an advantage. Often damage in one part of a body contributes to or causes damage in others. Being able to prevent that sort of damage through earlier transplant would be helpful.

### CONCLUSION, RAMIFICATIONS AND SCOPE

Thus, the reader will see that this invention of a multi-tiered model of immune system function provides answers to previously unaddressed questions, provides a framework for further research and provides direction for prevention screening, testing and treatment methods for a wide variety of IDO producing diseases and conditions.

Since this model spells out how the immune system actions are limited to only those targets perceived as Different/Not a Baby, treatments using this method would involve immune system attack directed at only specific targets, that is the ones where treatment has turned off the “Baby Sign”. Because other cells are not being attacked, these treatments are safer and less damaging than current treatments.

Further, since many of the choices available for use in these treatments are antioxidants, the treatments are readily available, safe and inexpensive.

While my above description has referred to the example of cancer treatment and the use of Ester-C and Vitamin D3, these should not be construed as limitations on the scope of the invention, but rather as an exemplification of one preferred embodiment thereof Many other variations are possible.

Further, the order of Treatment Choices as listed is not to be seen as the only order in which the choices may be made. For instance, one may both choose and start Treatment Part C before one chooses or starts Treatment Parts A and/or B. Again, many other variations are possible.

Accordingly, the scope of the invention should be determined not by the embodiments illustrated, but by the appended claims and their legal equivalents.

I claim:

1. A method of treating diseases and/or conditions associated with raised levels of the enzyme, indoleamine 2,3-dioxygenase in mammals by using a multi-tiered immune system model to direct specific immune system action upon said disease and/or condition, comprising:
(a) administering one or more types of antioxidants alone or in combination with one or more methods of stopping the production of, competing with, inhibiting, blocking, or neutralizing said enzyme or its actions whereby allowing the immune system to determine it does not need to treat said diseases and/or conditions as a child and, so, direct specific immune system action upon said disease and/or condition, and
(b) administering, if needed, a method or methods of building up and/or supporting the mammal's immune system, and
(c) administering, if needed, a method or methods of treating said diseases and/or conditions or their effects that does not detsroy, harm, diminish or interfere with the mammal's immune system or its function.

2. The method of claim 1 wherein the method of treatment part a comprises administering one or more types of antioxidants to compete with said enzyme for O2.

3. The method of claim 1 wherein the method of treatment part a comprises administering one or more types of antioxidants alone or in combination with one or more methods or stopping the production of, competing with, blocking or neutralizing said enzyme or its actions.

4. The treatment of claim 1 wherein said diseases and/or conditions are any form of cancer.

5. The treatment of claim 1 wherein said diseases and/or conditions involve parasites.

6. The treatment of claim 1 wherein said diseases and/or conditions are viral or bacterial infections or diseases.

7. The treatment of claim 1 wherein said diseases and/or conditions involve genetic mutation.

8. The treatment of claim 1 wherein said diseases and/or conditions involve damaged cells.

9. The treatment of claim 1 wherein the goal of said treatment is to improve the quality of life for the patient by diminishing or eliminating the symptoms of serotonin deficiency including, but not limited to, depression, altered pain perception, and sleep problems whereby allowing the mammal to feel more like themselves instead of feeling sick or hurt.

10. The treatment of claim 1 wherein the goal of said treatment is to cure or stop said diseases and/or conditions in a mammal.

11. The treatment of claim 1 wherein the goal of said treatment is to shrink, diminish, or eliminate the areas of the body affected by said diseases and/or conditions and/or stop the spread of said diseases and/or conditions in said mammal.

12. The treatment of claim 1 wherein the goal of said treatment is to improve the mammal's blood markers for said diseases and/or conditions.

13. A method of testing for either screening or treatment progress of diseases and/or conditions associated with raised levels of the enzyme, indoleamine 2,3-dioxygenase in mammals comprising the following
(a) screening choices: lower-than-normal ascorbic acid blood serum levels, higher-than-normal bowel tolerance levels for ascorbic acid, lower-than-normal blood serum tryptophan levels, and/or higher-than-normal blood serum levels of the by-products of tryptophan catabolization and
(b) positive treatment-progress testing choices: ascorbic acid blood serum levels going up, bowel tolerance for ascorbic acid going down, tryptophan blood serum levels going up, and/or blood serum levels of the by-products of tryptophan catabolization going down.

14. A method of preventing or lowering the chance of getting certain diseases and/or conditions associated with raised levels of the enzyme, indoleamine 2,3-dioxygenase in mammals by administering one or more types of antioxidants at optimal levels to scavenge as much available O2 as possible and thereby prevent production of said enzyme or successfully compete with said enzyme resulting in specific immune system action upon said diseases and/or conditions before they can take hold.

15. A method for directing and conducting research regarding diseases and/or conditions associated with raised levels of the enzyme, indoleamine 2,3-dioxygenase in mammals by using an immune system model with at least two tiers of questions to determine whether or not the immune system will act to direct research into the treatment and prevention of said diseases and/or conditions and means by which specific immune system action can by turned on and off.

16. A method of treatment using an immune system model with at least two tiers including a first tier of same/different and a second tier of baby/not a baby to turn on production of the enzyme, indoleamine 2,3-dioxygenase at given sites to prevent specific immune system action at target areas including, but not limited to, transplanted organs.
